(19) United States
(12) Patent Application Publication
(10) Pub. No.: US 2022/0117498 A1
(43) Pub. Date: Apr. 21, 2022

(54) SYSTEM FOR ASSISTING TRAINING AND METHOD THEREOF

(71) Applicant: PALPITO INC., Seongnam-si (KR)

(72) Inventor: Hyung Gon KIM, Yongin-si (KR)

(21) Appl. No.: 17/116,036

(22) Filed: Dec. 9, 2020

(30) Foreign Application Priority Data
Oct. 19, 2020 (KR) 10-2020-0135119
Nov. 12, 2020 (KR) 10-2020-0150712

(51) Int. Cl. A61B 5/024; A61B 5/11; A61B 5/00

(52) U.S. Cl. CPC A61B 5/024; A61B 5/1118; A61B 5/746; A61B 5/7405; A61B 5/742

(57) ABSTRACT

A system for assisting training and method thereof. A system for assisting training include a heart rate monitor to measure a user's heart rate; and a user terminal configured to receive a current heart rate measured by the heart rate monitor, to determine an exercise level and a duration according to the current heart rate, and to display a game content including a user character interlocked with the exercise level. The user terminal displays the exercise level divided into a plurality of sections so that the user character is positioned on an exercise level section corresponding to the current heart rate or the current heart rate and a user's location information and pays differential rewards according to at least one of the exercise level and the duration of the exercise level, in which the reward includes a reward for a game character or provision of a previously requested sound source.

### TECHNICAL FIELD

The present disclosure relates to a system for assisting training and method thereof.

### BACKGROUND

Enjoying a healthy life is human's old desire, and for this, humans have been making various efforts. In particular, various types of diet exercise methods have recently emerged to control weight or manage body shape through various exercises. However, even if people exercise, but they fail frequently because it is difficult to feel a sense of accomplishment since changes in their body are not as fast as they can see.

Therefore, various methods have been tried for sustainable exercise management. As an example, as part of a method of assisting exercise, there have been disclosed methods of calculating and providing calories consumed by exercisers or measuring a user's amount of exercise.

However, with these methods people may experience the effect of dieting in the early stages of exercise, but as exercise time accumulates, there is a problem that the effect cannot be felt with the same amount of exercise as in the past. In addition, even if data on the amount of exercise is accumulated and provided, if it lacks user's convenience, it may not provide meaningful data and also may not motivate users. Therefore, there is a need for measures of inducing users to do effective exercise while continuing exercise through appropriate motivation.

### SUMMARY

Various aspects of the present invention are directed to providing a system for assisting training and method thereof capable of continuously maintaining an exercise level that maximizes a user's exercise effect.

In addition, an embodiment of the present invention provides a system for assisting training and method thereof capable of visually providing the current exercise level to users to induce interest and improve exercise effect.

In addition, an embodiment of the present invention provides a system for assisting training and method thereof capable of supporting users to continue exercise with a sense of purpose by motivating users for exercise.

The technical problems to be solved in the present invention are not limited to the technical problems mentioned above, and other technical problems not mentioned will be clearly understood by those of ordinary skill in the art from the following description.

Various aspects of the present invention are directed to provide a system for assisting training, including: a heart rate monitor to measure a user's heart rate; and a user terminal configured to receive a current heart rate measured by the heart rate monitor, to determine an exercise level and a duration according to the current heart rate, and to display a game content including a user character interlocked with the exercise level, in which the user terminal displays the exercise level divided into a plurality of sections so that the user character is positioned on an exercise level section corresponding to the current heart rate or the current heart rate and a user's location information and pays differential rewards according to at least one of the exercise level and the duration of the exercise level, in which the reward includes a reward for a game character or provision of a previously requested sound source.

In an embodiment, the exercise level section may be a section in which exercise intensity is sorted by heart rate according to the amount of fat and carbohydrate consumed during exercise and is determined based on a target heart rate for each user and a section constant, wherein the target heart rate may be determined according to the user's age, exercise intensity, and difficulty level, wherein the section constant is a ratio to the target heart rate for dividing the exercise level sections into a plurality of exercise level sections.

In an embodiment, the plurality of exercise level sections may be divided into five stages of a first exercise level section to a fifth exercise level section, wherein the first exercise level section may be a section in which the current heart rate is 50% or more and less than 60% of the target heart rate, the second exercise level section may be a section in which the current heart rate is 60% or more and less than 70% of the target heart rate, the third exercise level section may be a section in which the current heart rate is 70% or more and less than 80% of the target heart rate, the fourth exercise level section may be a section in which the current heart rate is 80% or more and less than 90% of the target heart rate, and the fifth exercise level section may be a section in which the current heart rate is 90% or more of the target heart rate.

In an embodiment, the user terminal may pay the reward differentially according to a time maintained in the third exercise level section.

In an embodiment, the user terminal may output a voice feedback including an exercise coaching guide and a supportive message according to at least one of the exercise level and the duration of the exercise level.

In an embodiment, the user terminal may record the user's exercise date, exercise time, change in the heart rate and average maximum heart rate in linked to the user's account and recommend an exercise level and a duration that the user should maintain to obtain the reward.

In an embodiment, the user terminal may display an enemy character on the game content and remove the enemy character when the user character is located in the same exercise level section as the enemy character or remains in the same exercise level section for a predetermined time or longer.

In an embodiment, characteristics of the user character may change according to the exercise level.

In an embodiment, the heart rate monitor and the user terminal may be integrated or stand-alone wearable devices.

In an embodiment, the user terminal may determine a daily exercise target based on an exercise level and a duration of the exercise level, alarm the user when the user does not meet the daily exercise target, and provide a previously requested sound source when a summing up exercise target over a predetermined period is achieved.

In an embodiment, the user terminal may determine a corresponding exercise level by applying an exercise weighting according to a gradient and an altitude of a current location and recommend a remaining distance or an exercise path to achieve an exercise target from the current location.

In an embodiment, the user terminal may provide a sound source having a BPM corresponding to the heart rate of a corresponding exercise level and based on a maximum fat burning efficiency level among the exercise levels, provide the sound source slower than the original BPM of the sound source at an upper exercise level and provide the sound source faster than the original BPM of the sound source at a lower exercise level.

Various aspects of the present invention are directed to provide a method for assisting training, including: determining, by a user terminal, a target heart rate and an exercise level section; measuring, by a heart rate monitor, a heart rate; determining, by the user terminal, an exercise level and a duration according to the current heart rate measured by the heart rate monitor; displaying a game content including a user character interlocked with the exercise level that is divided into a plurality of sections so that the user character is positioned on an exercise level section corresponding to the current heart rate or the current heart rate and a user's location information; and paying differential rewards according to at least one of the exercise level and the duration of the exercise level, wherein the reward includes a reward for a game character or provision of a previously requested sound source.

In an embodiment, the method for assisting training may further include outputting a voice feedback including an exercise coaching guide and a supportive message according to at least one of the exercise level and the duration of the exercise level.

In an embodiment, the paying differential rewards may include paying, by the user terminal, the rewards differentially according to a time maintained at a maximum fat burning efficiency level among the exercise levels.

In an embodiment, the method for assisting training may further include recommending an exercise level and a duration that the user should maintain to obtain the reward.

A system for assisting training and method thereof according to an embodiment of the present invention may induce a user to continuously maintain an exercise level that maximizes an exercise effect by paying rewards for the user to exercise continuously at a maximum fat burning efficiency exercise level, thereby ensuring a definite exercise effect with only a minimum time and amount of exercise.

In addition, the present invention may visually provide an exercise level to induce interest and improve an exercise effect by displaying a game content including a user character interlocked with the exercise level, thereby inducing the user to be immersed in exercise without being bored.

In addition, the present invention may support a user to continuously exercise by motivating the user for exercise such as by paying rewards according to an exercise level and a duration and recommending an exercise level and a duration for obtaining the reward, thereby ensuring the continuity of the exercise.

### BRIEF DESCRIPTION OF THE DRAWINGS

The above and other aspects, features, and advantages of the present invention will become more apparent to those of ordinary skill in the art by describing embodiments thereof in detail with reference to the accompanying drawings, in which:

FIG. 1 is a block diagram illustrating a system for assisting training according to an exemplary embodiment of the present invention.

FIG. 2 is a block diagram illustrating a detail constitution of a user terminal of a system for assisting training according to an exemplary embodiment of the present invention.

FIG. 3 is a graph illustrating a heart rate over time according to general exercise.

FIG. 4 is a graph illustrating a heart rate over time according to exercise through a system for assisting training according to an exemplary embodiment of the present invention.

FIG. 5 is an example of a screen of a user terminal illustrating a game content of a system for assisting training according to an exemplary embodiment of the present invention.

FIG. 6 is another example of a screen of a user terminal illustrating a game content of a system for assisting training according to an exemplary embodiment of the present invention.

FIG. 7 is a further example of a screen of a user terminal illustrating a game content of a system for assisting training according to an exemplary embodiment of the present invention.

FIG. 8 is a diagram illustrating a start screen of the user terminal of FIG. 1.

FIG. 9 is a diagram illustrating a user information registration screen of the user terminal of FIG. 1.

FIG. 10 is a diagram illustrating a game selection screen of the user terminal of FIG. 1.

FIG. 11 is a diagram illustrating a voice feedback setting screen of the user terminal of FIG. 1.

FIG. 12 is a diagram for describing provision of sound sources according to an exercise level of a system for assisting training according to an exemplary embodiment of the present invention.

FIG. 13 is a diagram for describing application of an exercise weighting according to a position of a system for assisting training according to an exemplary embodiment of the present invention.

FIG. 14 is a diagram for describing a destination or a path recommendation according to a position of a system for assisting training according to an exemplary embodiment of the present invention.

FIG. 15 is a flowchart of a method for assisting training according to an exemplary embodiment of the present invention.

### DETAILED DESCRIPTION OF THE EMBODIMENTS

Hereinafter, exemplary embodiments of the present invention will be described in detail so that those of ordinary skill in the art can readily implement the present invention. The present invention may be embodied in many different forms and are not limited to the embodiments set forth herein. In the drawings, parts unrelated to the description are omitted for clarity. Throughout the specification, like reference numerals denote like elements.

Embodiments of the present invention are provided to describe the present invention more fully to those skilled in the art, the embodiments described below can be modified into various other forms, and the scope of the present invention is not limited to the following embodiments. Rather, these embodiments make the invention more meaningful and complete and are provided for fully conveying the concept of the invention to those skilled in the art.

Hereinafter, the embodiments of the present invention are described with reference to the drawings schematically illustrating the embodiments of the present invention. In the drawings, for example, variations in the illustrated shape may be expected depending on manufacturing techniques and/or tolerances. Accordingly, the embodiments of the present invention should not be construed as being limited to any particular shape of the regions illustrated herein and should include, for example, variations in shape resulting from manufacturing.

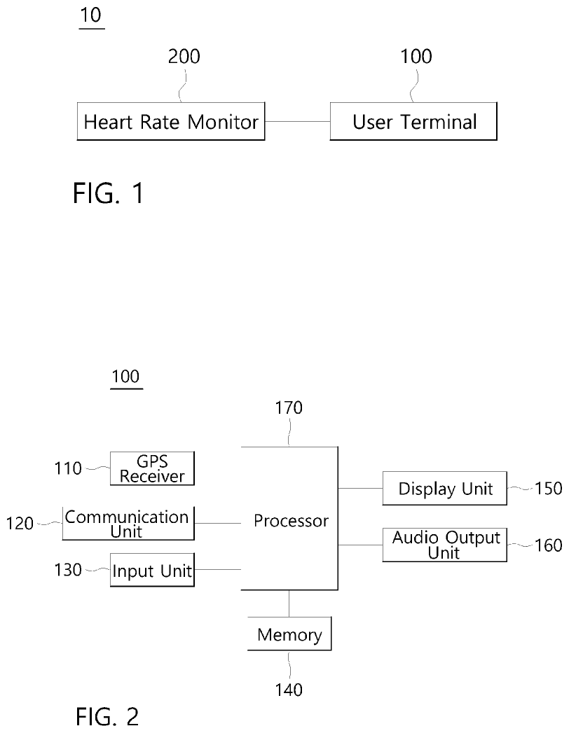

FIG. 1 is a block diagram illustrating a system for assisting training according to an exemplary embodiment of the present invention.

Referring to FIG. 1, in an embodiment, a system for assisting training 10 according to an embodiment of the present invention may include a user terminal 100 and a heart rate monitor 200.

The system for assisting training 10 may determine a level or intensity of exercise performed by a user from the user's heart rate, pay differential rewards for a game according to the exercise level, and provide different voice feedback based on some criteria such as the exercise level, intensity change, and duration.

The user terminal 100 is a device that receives a user's current heart rate from the heart rate monitor 200, determines an exercise level according to the current heart rate, and controls a game content corresponding thereto.

In an example, the user terminal 100 may determine an exercise level and a duration according to the current heart rate. In addition, the user terminal 100 may display a game content including a user character interlocked with the exercise level. In addition, the user terminal 100 may pay differential rewards according to at least one of the exercise level and the duration of the exercise level. In addition, the user terminal 100 may output a voice feedback according to at least one of the exercise level and the duration of the exercise level.

The user terminal 100 may be a device that is interlocked with the heart rate monitor 200 through a network, for example, an every kind of wireless communication device such as a smartphone, a smart pad, a tablet PC, and a personal communication system (PCS), global system for mobile communication (GSM), personal digital cellular (PDC), personal handy phone system (PHS), personal digital assistant (PDA), international mobile telecommunication (IMT) 2000, code division multiple access (CDMA) 2000, wideband-code division multiple access (W-CDMA) 2000, and wireless broadband Internet (WiBro) terminals, and a fixed terminal such as a desktop computer and a smart TV.

The heart rate monitor 200 may be worn by a user to measure the user's heart rate. For example, the heart rate monitor 200 may be a wearable device worn on a user's wrist such as a smart band and a smart watch. In addition, the heart rate monitor 200 may include a heart rate sensor to measure the user' heart rate and the heart rate sensor may use a photo plethysmograph (PPG) measurement method.

In this case, heart rate monitor 200 may include a separate storage unit to store the user's heart rate information and to transmit such heart rate information using a communication unit to the user terminal 100 connected through a network to the heart rate monitor 200.

In an embodiment, the user terminal 100 may be a wearable device that can be worn by a user independently of the heart rate monitor 200. Alternatively, the user terminal 100 may be integrally formed with the heart rate monitor 200. In this case, the user terminal 100 and the heart rate monitor 200 may be an integrated wearable device.

FIG. 2 is a block diagram illustrating a detail constitution of a user terminal of a system for assisting training according to an exemplary embodiment of the present invention.

Referring to FIG. 2, in an embodiment, the user terminal 100 may include a GPS receiver 110, a communication unit 120, an input unit 130, a memory 140, a display unit 150, an audio output unit 160 and a processor 170.

The GPS receiver 110 may receive GPS information from a Global Positioning System (GPS) satellite. Here, the GPS information may include topography and altitude information according to a user's current location. In addition, the GPS receiver 110 may store received GPS information in the memory 140.

The communication unit 120 may perform communication with the heart rate monitor 200. Here, the communication unit 120 may perform communication with the heart rate monitor 200 through a network including, but not limited to, a 3rd Generation Partnership Project (3GPP) network, a Long Term Evolution (LTE) network, a 5G network, a World Interoperability for Microwave Access (WiMAX) network, an Internet, a local area network (LAN), a wireless local area network (Wireless LAN), a wide area network (WAN), a personal area network (PAN), a Bluetooth network, a satellite broadcasting network, an analog broadcasting network, a digital multimedia broadcasting (DMB) network.

The input unit 130 may be a keyboard, a touch keypad, or a touch pad as a device through which a user's selection is input. In this case, the input unit 130 may be inputted, by a user, information for registering user information and calculating an exercise level.

The memory 140 may store heart rate information received from the heart rate monitor 200 and location information received from the GPS receiver 110. In addition, the memory 140 may store sound sources downloaded as a game content and a reward.

The display unit 150 may display a game content interlocked with an exercise level according to a user's current heart rate. In addition, the display unit 150 may display information on a series of operation states and operation results that occur while performing a function of the user terminal 100. In addition, the display unit 150 may display a menu of the user terminal 100 and user data input by the user. For example, the display unit 150 may include a liquid crystal display (LCD), a thin film transistor LCD (TFT-LCD), an organic light emitting diodes (OLED), a light emitting diode (LED), an active matrix organic LED (AMOLED), a flexible display, and a 3D display. Optionally, the display unit 150 may be a touch screen integrally formed with the input unit 130.

The audio output unit 160 may output a music using downloaded sound sources or a voice feedback according to an exercise level. In an example, the audio output unit 160 may be a speaker built into the user terminal 100. In another example, the audio output unit 160 may be a Bluetooth headset that receives and outputs sound or voice from the user terminal 100 through wireless communications.

The processor 170 may be configured to be communicatively connected to the GPS receiver 110, the communication unit 120, the input unit 130, the memory 140, the display unit 150, and the audio output unit 160 and to control them.

The processor 170 may control the display unit 150 to display an exercise level according to the current heart rate measured by the heart rate monitor 200 by dividing the exercise level into a plurality of sections. Here, the user character may be displayed on the display unit 150 to be positioned on an exercise level section corresponding to the current heart rate or the current heart rate and a user's location information. In addition, the user's location information may be an altitude, and a weighting of an exercise level as a gradient of a road.

In this case, the exercise level section may be a section in which exercise intensity is sorted by heart rate according to the amount of fat and carbohydrate consumed during exercise, and may be determined based on a target heart rate for each user and a section constant. Here, the target heart rate may be determined based on a user's age, exercise intensity, and difficulty level. The section constant may be a predetermined constant based on statistical data, and may be a ratio to the target heart rate for dividing the exercise level sections into a plurality of exercise level sections. As an example, the section constant may be a percentage of the heart rate.

More specifically, the target heart rate may be a set of criteria for guiding an exercise effective for body fitness to be performed. The exercise intensity represents an intensity of an exercise that a user intends to perform, and the processor 170 may receive the exercise intensity from the user's input at the input unit 130. As an example, the processor 170 may receive an input selected by the user from 80%, 90%, and 100% exercise intensity. In addition, the difficulty level is a difficulty level of exercise that the user intends to perform, and the processor 170 may receive the difficulty level from a user input. As an example, the processor 170 may receive an input selected by the user from a beginner's and intermediate difficulty level.

In this case, the processor 170 may be configured to calculate the heart rate in different ways according to the beginner's and intermediate difficulty levels. The target heart rate at the beginner's difficulty level may be calculated based on a user's age and an exercise intensity to be performed, and the target heart rate at the intermediate difficulty level may be calculated by further considering the user's maximum heart rate and resting heart rate as well as the user's age and the exercise intensity. First, the target heart rate at the beginner's difficulty level may be calculated using Equation 1 below.

Target heart rate=(220−age)×exercise intensity (%) [Equation 1]

Here, the number 220 may be a constant set to calculate the target heart rate, and the target heart rate may be regarded as the maximum heart rate when the exercise intensity is 100%. As an example, when a 31-year-old user selects the beginner's difficulty level and an exercise intensity of 80%, the processor 170 may draw a target heart rate of 151 bpm by putting the user's parameters into Equation 1 ((220−31)×0.8).

The target heart rate at the intermediate difficulty level, the target heart rate may be calculated using Equation 2 below.

Target heart rate=[(maximum heart rate−resting heart rate)×exercise intensity (%)]+resting heart rate [Equation 2]

Here, the maximum heart rate may be a value obtained by subtracting the user's age from the constant 220, and the resting heart rate may be an average heart rate calculated by measuring the user's heart rate for a preset time, for example, 1 minute by the heart rate monitor 200. As an example, when a 31-year-old user selects the intermediate difficulty level and an exercise intensity of 80%, the processor 170 may draw a target heart rate of 167 bpm by putting the user's parameters into Equation 2 as follows: [(((220−31)−80)×0.8)]+80, (for example, it is assumed the user's resting heart rate is 80). As described above, even for the same user, the target heart rate may be calculated differently according to whether to select beginner's or intermediate level. Accordingly, a user may set more effective target heart rate by selecting a difficulty level suitable for their level. However, the present invention is not limited thereto, and the equation for calculating the target heart rate may not change depending on the difficulty level.

In this case, the processor 170 may determine and recommend an exercise intensity and/or a section constant based on an accumulated and stored exercise data, exercise ability, exercise period, and exercise history of a user. Therefore, a plurality of aerobic exercise fat burning reference sections may be optimized and set differently for each user.

Here, a plurality of exercise level sections may be a plurality of aerobic exercise fat burning reference sections. As an example, a plurality of exercise level may be divided into five stages of a first exercise level section to a fifth exercise level section according to a proximity ratio to a target heart rate.

The first exercise level section may be a section in which the current heart rate is 50% or more and less than 60% (that is, the section constant is 0.5 to 0.6) of the target heart rate. The first exercise level section is a lowest area in which the metabolism of physical exercise begins, and may be a starting point of exercise for beginners or those with lowered physical strength since it places little burden on the body. In the first exercise level section, fat consumption may be high, but the amount of fat consumption may be slight since overall amount of exercise is small.

The second exercise level section may be a section in which the current heart rate is 60% or more and less than 70% (that is, the section constant is 0.6 to 0.7) of the target heart rate. This section has an intensity capable of having a conversation with a person next to the user, and may consume more calories compared to the first exercise level section. The second exercise level section may be a point in which sweating begins slightly and fat burning in the cells begins for losing weight, so it may be regarded as an area for losing weight.

The third exercise level section may be a section in which the current heart rate is 70% or more and less than 80% (that is, the section constant is 0.7 to 0.8) of the target heart rate. Here, the third exercise level section may be referred to as an aerobic or sweet zone, and may be an oxygen consumption area in which a lot of sweat comes out. When exercising while maintaining the 3rd exercise level section, the user may see an effect of improvement physical strength thanks to improvement of cardiopulmonary capacity, and those who enjoy aerobic exercise may experience a feeling of happiness also referred to as a state of runner's high since more calories are consumed compared to the previous exercise level sections, leading to a secretion of endorphins.

The fourth exercise level section may be a section in which the current heart rate is 80% or more and less than 90% (that is, the section constant is 0.8 to 0.9) of the target heart rate. This section may be an area that contains the meaning of an important threshold for exercise. Exercising about twice a week in this area may be very helpful in improving quickness and overall exercise ability, but keeping this area too much tends to overtrain.

The fifth exercise level section may be a section in which the current heart rate is 90% or more (that is, the section constant is 0.9 or more) of the target heart rate. This section may be a high intensity training area and a professional training area. In the fifth exercise level section, body temperature rises a lot, exercise may be hard and pain may be felt, and when the condition is good and the body is healthy, it gives a lot of stimulation to physical development, but failure to adjust pace shows a high tendency to overtrain.

Referring to examples described above, when a 31-year-old user sets an exercise level of 80% in the beginner difficulty level, the user terminal 100 may draw a target heart rate of 151 bpm. In this case, the heart rate of the first exercise level section may be determined as 75 to 90 bpm since it is 50% to 60% of the target heart rate; the heart rate of the second exercise level section may be determined as 90 to 105 bpm since it is 60% to 70% of the target heart rate; the heart rate of the third exercise level section may be determined as 105 to 120 bpm since it is 70% to 80% of the target heart rate; the heart rate of the fourth exercise level section may be determined as 120 to 135 bpm since it is 80% to 90% of the target heart rate; and the heart rate of the fifth exercise level section may be determined as 135 bpm or more since it is 90% or more of the target heart rate. As described above, since the exercise level section may be determined based on the user's target heart rate, an optimized exercise level section for each user may be determined. In addition, since the second exercise level section and the third exercise level section, in which fat burning is the most efficient and not excessive, are also optimized for each user, information on the optimal exercise intensity for body fitness may be provided to the user, and an exercise intensity for preventing overtraining may be recognized by the user by using a plurality of exercise level sections determined through the user's own target heart rate.

In addition, the processor 170 may be configured to pay differential rewards according to at least one of the exercise level and the duration of the exercise level. Here, the reward may be a reward for a game character displayed on the display unit 150. Optionally, the processor 170 may pay differential rewards for a game content linked to a user account. In addition, the reward may include providing a previously requested sound source. In this way, the processor 170 may allow the user to select a reward.

The processor 170 may control the audio output unit 160 to output a voice feedback including an exercise coaching guide and a supportive message according to at least one of an exercise level and a duration of the exercise level.

As an example, the processor 170 may generate a first voice feedback when the heart rate enters the second exercise level section among the plurality of exercise level sections. Entering the second exercise level section may mean that the warm-up exercise is completed and full-scale exercise begins. For example, the first voice feedback may be “The warm-up is over. From now, you are in aerobic exercise mode. Fat burning will start from now.” In addition, the processor 170 may generate a second voice feedback when an exercise level corresponding to the heart rate is lower than the second exercise level section. The second voice feedback may be generated in case an exercise level corresponding to the heart rate enters the second exercise level section and is lower than the second exercise level. In an example, the second voice feedback may be “From now on, aerobic exercise mode will be released”, which may help the user to figure out effective fat burning time and to adjust the exercise intensity.

In addition, the processor 170 may generate a third voice feedback as an indication of maintaining a pace when the third exercise level section is maintained for a predetermined time. For example, the third voice feedback may be “Fighting!! You are doing well,” which may help keep the user in a state of runner's high where endorphins are released, thus maintaining the user's running pace.

In addition, the processor 170 may generate a fourth voice feedback when entering the fourth exercise level section. For example, the fourth voice feedback may be “Please lower the pace a little,” which may induce the user to lower the pace so as not to be injured or lose interest due to over pace.

In addition, the processor 170 may generate a fifth voice feedback when failing to enter the second exercise level section for a predetermined time, for example 3 minutes, or failing to enter the third exercise level section from the second exercise level section and stayed in the second exercise level section. For example, the fifth voice feedback may be “Please increase your pace a little.”

Various voice feedbacks by the processor 170 described above, in one embodiment, may provide different voice feedbacks each time entering an upper or lower exercise level section and/or when a certain time elapses in a specific exercise level section, thereby improving exercise efficiency and motivation of the user. Users may perform more intuitively effective exercise because they hear corresponding voice feedback based on the heart rate that fits their condition.

In this case, the processor 170 may provide the payment of game content reward at the same time as the voice feedback. In an example, when a user's heart rate is maintained for 3 to 5 minutes at the third exercise level, the third voice feedback may be provided along with the payment of additional reward. Therefore, even when it is difficult for the user to check their exercise state since they concentrate on the game, the user may check their exercise state effectively since the user can hear the voice feedback through the audio output unit 160.

In addition, the processor 170 may pay differential rewards for game based on the type and number of voice feedbacks and the type and duration of a section in which the heart rate is maintained. For example, comparing when receiving 1 first voice feedback and 1 third voice feedback and maintaining a total of 5 minutes or more at the third exercise level (a first case) with when receiving 1 first voice feedback, 1 third voice feedback, 1 fourth voice feedback and 1 second voice feedback and maintaining a total of 5 minutes or more at the third exercise level (a second case), even if both the first case and the second case have the same duration, the second case may have less rewards for game content compared to the first case in which the exercise state is maintained stable at the third exercise level since the second case is a state with severe fluctuations in the exercise state compared to the first case.

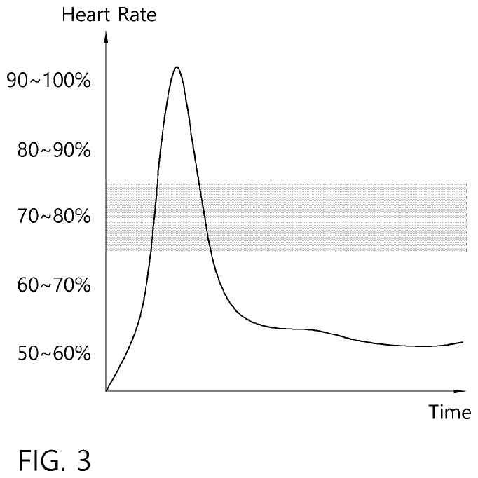

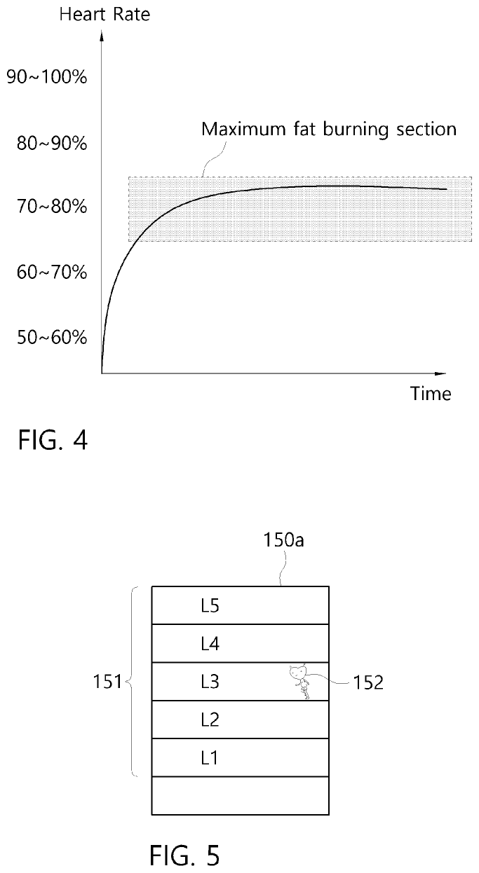

FIG. 3 is a graph illustrating a heart rate over time according to general exercise and FIG. 4 is a graph illustrating a heart rate over time according to exercise through a system for assisting training according to an exemplary embodiment of the present invention.

Referring to FIG. 3, for a general exercise, the heart rate increases rapidly at the beginning of the exercise and then changes to a stable state as a user becomes more familiar with the exercise. That is, the change in heart rate rapidly rises at the beginning, and after reaching the maximum heart rate, it drops rapidly and remains constant. This can be understood as a section in which the user catches their breath during the first exercise. It may be seen that the heart rate rises rapidly at the beginning of exercise, unnecessarily wasting physical strength, and exercise effects such as fat burning are not efficient during continuous exercise. Here, the heart rate is expressed as a section constant (percentage) with respect to the target heart rate.

Referring to FIG. 4, when exercising using a system for assisting training 10 according to an embodiment of the present invention, the heart rate may be continuously maintained after entering the maximum fat burning section at the beginning of the exercise. This indicates that the exercise effect may be maximized through continuous fat burning exercise without relatively large consumption of physical strength due to a higher exercise level. Here, the maximum fat burning section may be a third exercise level section L3 in which the heart rate is 70 to 80% of the maximum heart rate.

To this end, the processor 170 may pay the largest rewards for the third exercise level, and may pay additional differential rewards according to a time maintained in the third exercise level section.

Accordingly, the system for assisting training 10 according to an embodiment of the present invention may induce a user to continuously maintain an exercise level that maximizes an exercise effect, thereby ensuring a definite exercise effect with only a minimum time and amount of exercise.

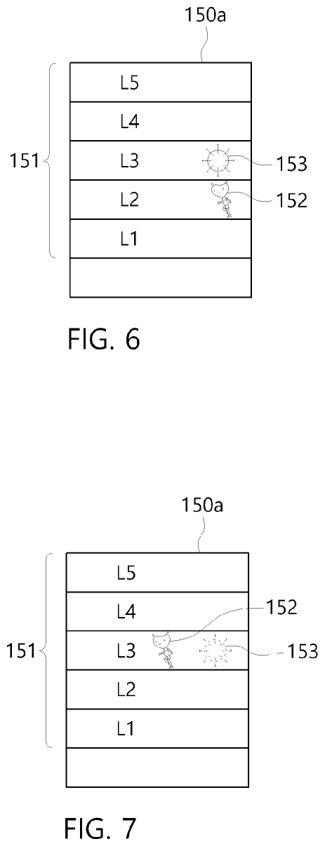

FIG. 5 is an example of a screen of a user terminal illustrating a game content of a system for assisting training according to an exemplary embodiment of the present invention, FIG. 6 is another example of a screen of a user terminal illustrating a game content of a system for assisting training according to an exemplary embodiment of the present invention, and FIG. 7 is a further example of a screen of a user terminal illustrating a game content of a system for assisting training according to an exemplary embodiment of the present invention.

Referring to FIG. 5, the processor 170 may control to display a plurality of exercise level sections 151 on a screen 150a. In this case, the exercise level section 151 may include five stages of a first exercise level section L1, a second exercise level section L2, a third exercise level section L3, a fourth exercise level section L4, and a fifth exercise level section L5.

The exercise level section 151 may be displayed so that the exercise level sections are distinguished from each other. For example, the first exercise level section L1 corresponding to the heart rate furthest from the target heart rate may be located at the bottom of the screen, and the fifth exercise level section L5 corresponding to the heart rate closest to the target heart rate may be located at the top of the screen.

In this case, a user character 152 may be positioned on the corresponding exercise level section 151 in association with the user's heart rate. Specifically, the processor 170 may receive a user's heart rate information from the heart rate monitor 200 and determine an exercise level section corresponding thereto. Here, the processor 170 may control the user character 152 to be positioned in a specific exercise level section among the plurality of exercise level sections 151 by interlocking the user character 152 with the user's heart rate.

In an example, when a user's heart rate is 110 bpm, this corresponds to the third exercise level section, so the user character 152 may be positioned in the third exercise level section L3 on the screen 150a as shown in FIG. 5. In this state, when the heart rate reaches 100 bpm as the user decreases his/her exercise pace, the user character 152 may be moved to the second exercise level section L2. In this way, the user character 152 may be interlocked to the user's heart rate in real time, the height may be displayed differently according to a relatively high or low heart rate even in the same exercise level section 151.

Accordingly, the system for assisting training 10 according to an embodiment of the present invention may maximize the exercise effect by increasing motivation and inducing interest by providing the user with a game interlocked to the heart rate.

On the other hand, when only the location of the user character 152 is displayed on the screen 150a, the user may have insufficient motivation for exercise. Therefore, in order to compensate for this, the processor 170 may control the game content to provide a reward such as “acquiring ice cream.”

Specifically, the processor 170 may control the game content linked to a user account to pay rewards according to an exercise level corresponding to a heart rate and a duration of the exercise level. In an example, when an exercise level corresponding to a heart rate is maintained for more than a predetermined time in the second exercise level or the third exercise level (when the user character 152 is maintained for more than a predetermined time in the second exercise level section L2 or in the third exercise level section L3 on the screen 150a), the processor 170 may control the game content linked to a user account to provide additional rewards.

In addition, the processor 170 may pay differential rewards for the game content according to a time during which the exercise level corresponding to the heart rate is maintained in the second exercise level or in the third exercise level. In an example, the processor 170 may provide rewards after entering the first exercise level, and when the exercise continues to enter the second exercise level or the third exercise level, it may provide more rewards than the reward given in the first exercise level and it may provide more rewards as the time maintained in the second exercise level or in the third exercise level increases. In addition, when the second exercise level or the third exercise level is maintained for more than a predetermined time, an additional reward may be provided in addition to the rewards above. In addition, the processor 170 may recommend an exercise level and a duration that the user should maintain in order to obtain an additional reward for the game content, and provide it through the screen 150a.

In this way, the system for assisting training 10 may motivate the user to continue to exercise in the second exercise level or in the third exercise level, which is a maximum fat burning efficiency section, by paying differential rewards according to the heart rate of the user and by providing information for obtaining additional rewards.

Accordingly, the system for assisting training 10 according to an embodiment of the present invention not only pays rewards for a game when simply entering an exercise target section (for example, the second exercise level or the third exercise level), but also it increases rewards for a game as the time maintained in the corresponding section increases. So, it may motivate the user to continuously maintain the optimal exercise state to assist the user to continue to exercise, thereby ensuring the continuity of the exercise.

In this case, such rewards may be items such as skills, special moves, weapons that can be used in the game, or may be rewards for levels, experience points, scores, and in-game goods, etc. Alternatively, in a different way, such rewards may be a concept of gift certificates or exchange vouchers that allow the user to purchase goods at convenience stores or the like outside of the game.

Meanwhile, the processor 170 may control to record a user's exercise date, exercise time, change in the heart rate and average maximum heart rate in linked to the user's account. The user may check the amount of exercise of the user and the degree of rewards of the game content obtained by the exercise of the user through the above-described exercise history recorded in linked to the user's account. Users may be not motivated only by data on their own amount of exercise, but may also be motivated through game content rewards, for example, the character of the game content that grows through experience points, so the self-efficacy of growing up with the character can be inspired.

Referring to FIG. 6, processor 170 may control to further provide an enemy character 153 to a game content.

Referring to FIG. 7, such an enemy character 153 may be positioned in a same exercise level section 151 as a user character 152 or may be removed when it is maintained in a same exercise level section 151 for a predetermined time.

In an example, when the current user's heart rate is 100 bpm, the user character 152 may be positioned in the second exercise level section L2 as shown in FIG. 6. In such a state, when the enemy character 153 appears in the third exercise level section L3, the user should increase their heart rate by increasing exercise pace in order to remove the enemy character 153 in a game. For example, when the heart rate reaches 110 bpm, as shown in FIG. 7, the user character 152 may be positioned in the third exercise level section L3 identical to the position of the enemy character 153. In this case, the enemy character 153 may be removed at the same time the user character 152 enters the third exercise level section L3 or after the user character is maintained in the third exercise level section L3 for a predetermined time.

Accordingly, the system for assisting training 10 according to an embodiment of the present invention may visually provide an exercise level to induce interest and improve an exercise effect, thereby inducing the user to be immersed in exercise without being bored.

In this case, in order to enhance the fun of the game, characteristics of the user character 152 such as an attack power or a defense power of the user character 152 may be possible to be changed according to the position of the user character 152 in the plurality of exercise level sections 151. For example, characteristics of the user character 152 may be changed such as: when the user character 152 is positioned in the third exercise level section L3, the attack power may be set to be the strongest, and when the user character 152 is positioned in the fourth exercise level section L4 or in the fifth exercise level section L5, the user character 152 may be set to use the special move. Accordingly, the user may be more immersed in the game, and may obtain improved exercise effect by passing through the various exercise level sections 151.

The processor 170 may determine a daily exercise target based on an exercise level and a duration of the exercise level. In an example, the daily exercise target may be determined according to another exercise level and duration based on the exercise target maintaining for 20 minutes at the third exercise level. In this case, the exercise target maintaining for 20 minutes at the third exercise level may be scored as 100 points, and based on this, another exercise level and a duration according to the exercise level may be scored. That is, the daily exercise target may be determined as 100 points in terms of scores.

In addition, the processor 170 may control to alarm to encourage the user to exercise more when the user does not meet the daily exercise target. In an example, the processor 170 may control to alarm through vibrations, sounds or messages.

In addition, the processor 170 may control to provide a previously requested sound source when a summing up exercise target over a predetermined period is achieved. In this case, a score obtained by summing each daily exercise target corresponding to a certain period such as 1 day, 1 week, and 1 month may be the exercise target for the period. In an example, if the period is 1 week, the summing up exercise target may be 700 scores. In this case, the user may receive a reward for achieving the summing up exercise target for a week, separate from the daily exercise target.

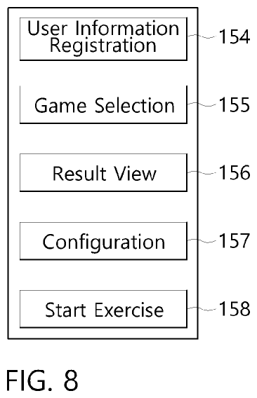

FIG. 8 is a diagram illustrating a start screen of the user terminal of FIG. 1.

Referring to FIG. 8, a start screen may be displayed when a training assist application is executed in the user terminal 100. On the start screen, a user information registration icon 154, a game selection icon 155, a result view icon 156, a configuration icon 157, and a start exercise icon 158 may be displayed.

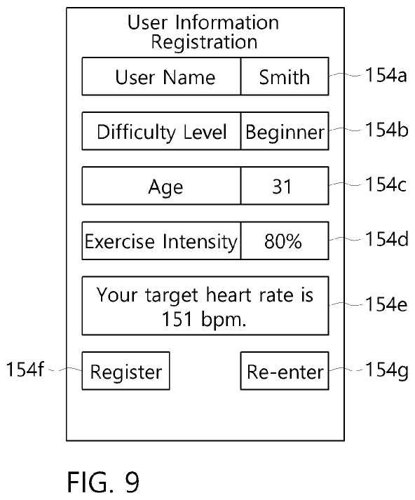

FIG. 9 is a diagram illustrating a user information registration screen of the user terminal of FIG. 1.

Referring to FIG. 9, when receiving a user input for the user information registration icon 154, the processor 170 may control the display unit 150 to display a screen for registering a user account. A user name may be input to a user name icon 154a, and a difficulty level may be selected from a difficulty level icon 154b. In addition, a user's age may be input to an age icon 154c, and an exercise intensity may be selected from an exercise intensity icon 154d.

Here, a target heart rate display window 154e may display a target heart rate calculated based on the difficulty level, age, and exercise intensity input by the user. In this case, when a register icon 154f is selected, the input user information may be stored in the memory 140 to complete the registration of the user account, and when a re-enter icon 154g is selected, the user name, difficulty level, and exercise intensity may be reset.

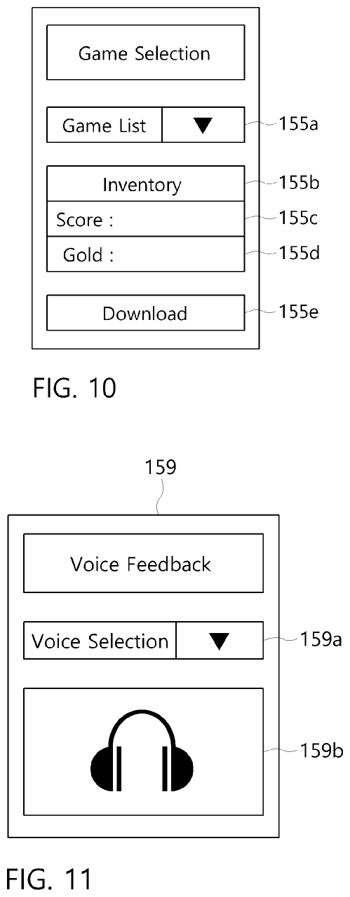

FIG. 10 is a diagram illustrating a game selection screen of the user terminal of FIG. 1.

Referring to FIG. 10, when a user input for the game selection icon 155 is received, the processor 170 may control the display unit 150 to display a game selection screen. One of a plurality of game contents may be selected from a game list icon 155a. An inventory icon 155b may display rewards acquired by the user from the game content selected by the user, for example, possession status of items.

In addition, a score display window 155c may display a score obtained by the user from the game content selected by the user. This score may be obtained when the reward obtained through exercise by the user is a score, and the type of reward may be different for each game content. When the reward for the game selected by the user is gold, that is, game money, a gold display window 155d may display game money obtained by the user.

A download icon 155e may be activated when the game content selected by the user is not installed in the user terminal 100. When receiving a user input for the download icon 155e, the processor 170 may control the game content to be downloaded from online.

FIG. 11 is a diagram illustrating a voice feedback setting screen of the user terminal of FIG. 1.

Referring to FIG. 11, when receiving a user input for a voice feedback icon, the processor 170 may control the display unit 150 to display the voice feedback setting screen 159. When a voice selection icon 159a is selected, a voice selection pop-up window may be displayed on the display unit 150. The user may select a favorite voice by clicking a selection icon for a desired voice among a plurality of voices. For example, the voice may include not only general mechanical sounds, but also voices of someone that the user likes such as entertainers, family members, and acquaintances.

In addition, when receiving a user input for a pre-listening icon 159b, the processor 170 may control to transmit a pre-listening voice for a voice selected by the user to the audio output unit 160.

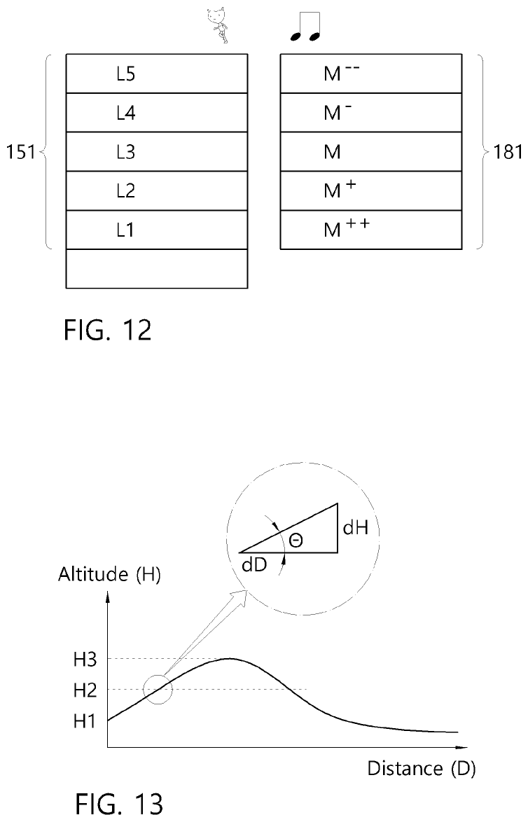

FIG. 12 is a diagram for describing provision of sound sources according to an exercise level of a system for assisting training according to an exemplary embodiment of the present invention.

Referring to FIG. 12, the processor 170 may control to provide a sound source having a BPM corresponding to a heart rate of a corresponding exercise level. Here, providing a sound source may include executing the sound source. That is, in the exercise level sections 151, a sound source having a BPM corresponding to each section may be played and output to the audio output unit 150.

In this case, based on the third exercise level which is the maximum fat burning efficiency level among multiple exercise levels, a sound source provided at an upper exercise level may be provided slower (M− or M−−) than an original BPM of a corresponding sound source, and a sound source provided at a lower exercise level may be provided faster (M+or M++) than an original BPM of a corresponding sound source.

Accordingly, the system for assisting training 10 according to an embodiment of the present invention may easily induce and manage the user's exercise pace adjustment since it may recommend to increase exercise pace by increasing the BPM in order to induce the user to move to the third exercise level section L3 when the user is exercising in the first exercise level section L1 or in the second exercise level section L2 and it may it may recommend to decrease exercise pace by decreasing the BPM in order to induce the user to move to the third exercise level section L3 when the user is exercising in the fourth exercise level section L4 or in the fifth exercise level section L5.

FIG. 13 is a diagram for describing application of an exercise weighting according to a position of a system for assisting training according to an exemplary embodiment of the present invention.

Referring to FIG. 13, a user's exercise path may include a different altitude H for each location. In this case, each location may have a gradient (φ). Here, the gradient (φ) may be expressed as a change in altitude (dH) with respect to a change in distance (dD).

The processor 170 may determine a corresponding exercise level by applying an exercise weighting according to a gradient (φ) and an altitude (H) of the current location. In an example, the exercise level may be determined to increase according to the altitude or gradient since the heart rate increases when the altitude (H) is high or the gradient (φ) is large.

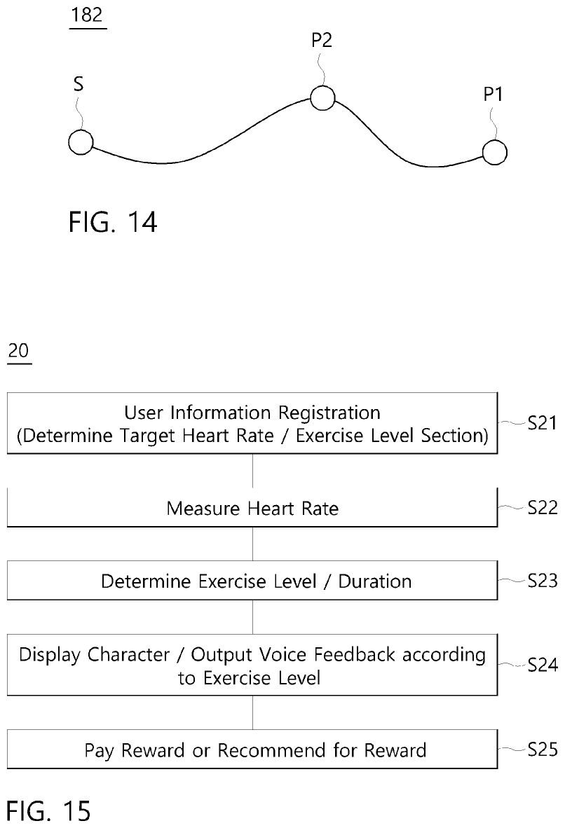

FIG. 14 is a diagram for describing a destination or a path recommendation according to a position of a system for assisting training according to an exemplary embodiment of the present invention.

Referring to FIG. 14, the processor 170 may recommend a remaining distance or an exercise path 182 to achieve an exercise target from a current location S. In an example, when exercising, the processor 170 may calculate a remaining distance, that is, a distance to the target point P2, to achieve the exercise target from the current location S and recommend it to the user. In this case, the remaining distance may be calculated in consideration of maintaining the current exercise level. Here, the processor 170 may calculate the remaining distance according to a remaining duration to achieve the exercise target based on the user's average speed with respect to the current exercise level.

In addition, when starting exercise, the processor 170 may calculate the exercise path 182 to the destination P1 and recommend it to the user. Here, the exercise path 182 may be calculated on the assumption that the third exercise level is maintained for a predetermined time. In an example, the processor 170 may calculate a path corresponding to a target distance by calculating the target distance (or a target point P1) according to a duration based on the user's average speed with respect to the third exercise level.

In this case, when the user's exercise level changes, the exercise path 182 to the target point P1 may be changed or the target point P1 on the exercise path 182 may be changed.

Hereinafter, a method for assisting training according to an embodiment of the present invention will be described with reference to FIG. 15.

FIG. 15 is a flowchart of a method for assisting training according to an exemplary embodiment of the present invention.

The method for assisting training 20 may include user information registration step (S21), heart rate measurement step (S22), exercise level/duration determination step (S23), character display step (S24) according to exercise level, and reward payment step (S25).

More specifically, as shown in FIG. 15, first, the user terminal 100 registers a user information at step S21. Here, the user information may include a user name, a difficulty level, a user's age, and an exercise intensity.

In this case, the user terminal 100 may determine a target heart rate and an exercise level section. Here, the target heart rate may be calculated based on the user's age, the exercise intensity and difficulty level of an exercise to be performed by the user. Exercise level sections may be a plurality of aerobic exercise fat burning reference sections. As an example, a plurality of exercise level may be divided into five stages of a first exercise level section to a fifth exercise level section according to a proximity ratio to a target heart rate.

Next, the heart rate monitor 200 measures the user's heart rate at step S22. In this case, the user may be a user previously registered in the system for assisting training 10, and the heart rate monitor 200 may transmit the measured heart rate to the user terminal 100.

Next, the user terminal 100 determines an exercise level and a duration according to the current heart rate measured by the heart rate monitor 200 at step S23. Here, the user terminal 100 may determine an exercise level determined in response to the current heart rate.

In addition, the user terminal 100 may determine the duration of the exercise level. Here, the duration may be a time during which a change in the heart rate is very small or even if there is a change a state in which the exercise level is not changed is maintained.

Next, the user terminal 100 displays a game content including a user character interlocked with the exercise level at step S24. In this case, the user terminal 100 may display the exercise level divided into a plurality of sections. Here, the user character may be displayed on the display unit 150 to be positioned on an exercise level section corresponding to the current heart rate or the current heart rate and a user's location information. The user's location information may include an altitude, and a gradient of a road.

In this case, the user terminal 100 may output a voice feedback according to at least one of the exercise level and the duration of the exercise level. Here, the voice feedback may include an exercise coaching guide and a supportive message. In an example, the user terminal 100 may output a first voice feedback to a fifth voice feedback that notifies or encourages an exercise state according to the exercise level and the duration.

Next, the user terminal 100 pays differential rewards according to at least one of the exercise level and the duration of the exercise level at step S25. Here, the reward may be a reward for the game character. Alternatively, the reward may be the provision of a user-selectable sound source. The sound source may be requested in advance.

In this case, the user terminal 100 may pay differential rewards according to a time maintained at the maximum fat burning efficiency level among the exercise levels. In an example, the user terminal 100 may pay the largest rewards for the third exercise level, and may pay additional differential rewards according to a time maintained in the third exercise level section.

In addition, the user terminal 100 may recommend an exercise level and a duration that the user should maintain to obtain a reward. In this case, the user terminal 100 may calculate how many levels in the exercise level or how much time in the duration of the exercise level the user should increase to obtain additional rewards during exercise at the current exercise level and recommend them to the user.

Such methods may be implemented by the system for assisting training 10 as shown in FIG. 1, and in particular, may be implemented as a software program that performs such steps, wherein such program may be stored on a computer-readable recording medium or transmitted by a computer data signal combined with a carrier wave in a transmission medium or a communication network. In this case, the computer-readable recording medium may include any kind of recording device in which data readable by a computer system is stored.

Although exemplary embodiments of the present invention have been described above, the spirit of the present invention is not limited to the embodiments set forth herein. Those of ordinary skill in the art who understand the spirit of the present invention may easily propose other embodiments through supplement, change, removal, addition, etc. of elements within the same spirit, but the embodiments will be also within the scope of the present invention.

What is claimed is:

1. A system for assisting training, comprising:
a heart rate monitor to measure a user's heart rate; and
a user terminal configured to receive a current heart rate measured by the heart rate monitor, to determine an exercise level and a duration according to the current heart rate, and to display a game content including a user character interlocked with the exercise level, wherein the user terminal displays the exercise level divided into a plurality of sections so that the user character is positioned on an exercise level section corresponding to the current heart rate or the current heart rate and a user's location information and pays differential rewards according to at least one of the exercise level and the duration of the exercise level, wherein the reward comprises a reward for a game character or provision of a previously requested sound source.

2. The system of claim 1, wherein the exercise level section is a section in which exercise intensity is sorted by heart rate according to the amount of fat and carbohydrate consumed during exercise and is determined based on a target heart rate for each user and a section constant, wherein the target heart rate is determined according to the user's age, exercise intensity, and difficulty level, wherein the section constant is a ratio to the target heart rate for dividing the exercise level sections into a plurality of exercise level sections.

3. The system of claim 2, wherein the plurality of exercise level sections are divided into five stages of a first exercise level section to a fifth exercise level section, wherein the first exercise level section is a section in which the current heart rate is 50% or more and less than 60% of the target heart rate, the second exercise level section is a section in which the current heart rate is 60% or more and less than 70% of the target heart rate, the third exercise level section is a section in which the current heart rate is 70% or more and less than 80% of the target heart rate, the fourth exercise level section is a section in which the current heart rate is 80% or more and less than 90% of the target heart rate, and the fifth exercise level section is a section in which the current heart rate is 90% or more of the target heart rate.

4. The system of claim 3, wherein the user terminal pays the reward differentially according to a time maintained in the third exercise level section.

5. The system of claim 1, wherein the user terminal outputs a voice feedback including an exercise coaching guide and a supportive message according to at least one of the exercise level and the duration of the exercise level.

6. The system of claim 1, wherein the user terminal records the user's exercise date, exercise time, change in the heart rate and average maximum heart rate in linked to the user's account and recommends an exercise level and a duration that the user should maintain to obtain the reward.

7. The system of claim 1, wherein the user terminal displays an enemy character on the game content and removes the enemy character when the user character is located in the same exercise level section as the enemy character or remains in the same exercise level section for a predetermined time or longer.

8. The system of claim 1, wherein characteristics of the user character change according to the exercise level.

9. The system of claim 1, wherein the heart rate monitor and the user terminal are integrated or stand-alone wearable devices.

10. The system of claim 1, wherein the user terminal determines a daily exercise target based on an exercise level and a duration of the exercise level, alarms the user when the user does not meet the daily exercise target, and provides a previously requested sound source when a summing up exercise target over a predetermined period is achieved.

11. The system of claim 1, wherein the user terminal determines a corresponding exercise level by applying an exercise weighting according to a gradient and an altitude of a current location and recommends a remaining distance or an exercise path to achieve an exercise target from the current location.

12. The system of claim 1, wherein the user terminal provides a sound source having a BPM corresponding to the heart rate of a corresponding exercise level and based on a maximum fat burning efficiency level among the exercise levels, provides the sound source slower than the original BPM of the sound source at an upper exercise level and provides the sound source faster than the original BPM of the sound source at a lower exercise level.

13. A method for assisting training, comprising:
determining, by a user terminal, a target heart rate and an exercise level section;
measuring, by a heart rate monitor, a heart rate;
determining, by the user terminal, an exercise level and a duration according to the current heart rate measured by the heart rate monitor;
displaying a game content comprising a user character interlocked with the exercise level that is divided into a plurality of sections so that the user character is positioned on an exercise level section corresponding to the current heart rate or the current heart rate and a user's location information; and
paying differential rewards according to at least one of the exercise level and the duration of the exercise level, wherein the reward comprises a reward for a game character or provision of a previously requested sound source.

14. The method of claim 13, further comprising:
outputting a voice feedback including an exercise coaching guide and a supportive message according to at least one of the exercise level and the duration of the exercise level.

15. The method of claim 13, wherein the paying differential rewards includes paying, by the user terminal, the rewards differentially according to a time maintained at a maximum fat burning efficiency level among the exercise levels.

16. The method of claim 13, further comprising:
recommending an exercise level and a duration that the user should maintain to obtain the reward.
